(19) United States
(12) Patent Application Publication
(10) Pub. No.: US 2008/0249325 A1
(43) Pub. Date: Oct. 9, 2008

(54) Method and Device For Continuous Process of Transesterification of Carboxylic Acid Esters in a Supercritical Monovalent Alcohol

(75) Inventor: Toomas Kaevand, Estonia (EE)

Correspondence Address: BIRCH STEWART KOLASCH & BIRCH, PO BOX 747, FALLS CHURCH, VA, 22040-0747, US

(73) Assignee: NBD Technologies OU, Tallinn (EE)

(21) Appl. No.: 10/590,718

(22) PCT Filed: Feb. 25, 2005
(86) PCT No.: PCT/EE2005/000003

§ 371 Date: Jun. 16, 2008

(30) Foreign Application Priority Data
Feb. 25, 2004 (EE) P200400060

(51) Int. Cl. C07C 67/02; C11C 3/04; B01J 8/04

(52) U.S. Cl. 554/167; 560/129; 422/196

(57) ABSTRACT

A method for continuous process of transesterification of carboxylic acid esters in supercritical monovalent alcohol, wherein, accordingly formula R—OH, R is discretionary aliphatic hydrocarbon radical having 1-10 carbon atoms and in which as initial substance are used synthetic carboxylic acid esters or their compositions, for example fats and oils, which are esters of different fatty acids and glycerol. A device for continuous process of transesterification of carboxylic acid esters in supercritical monovalent alcohol, which comprises alcohol inlet (1), carboxylic acid ester inlet (2), reactant dosing unit (3), heat-exchanger (4), transesterification unit (5), tube of hot reaction yield to expansion tank (6), expansion tank (7), tube of hot reaction yield to heat-exchanger (8), reverse current tube of excess of alcohol (9), tube of cooled reaction yield to separator (10), separator (11), outlets of new ester and new alcohol (12 and 13).

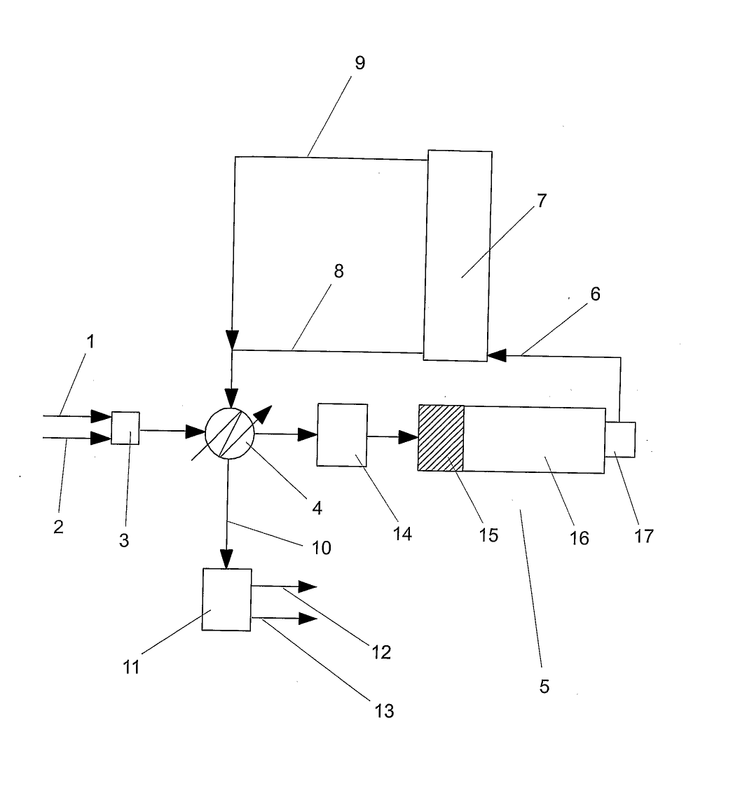

### TECHNICAL FIELD

Present invention belongs to the area of methods for industrial continuous process carboxylic acids transesterification in a supercritical monovalent alcohol.

### BACKGROUND ART

There are many different well-known carboxylic acid transesterification methods from patent documents as well as from scientific articles. Different catalysts have been used in majority of that type described reactions, catalysists used having been mainly the alkali (Schwab, A. W., Bagby, M. O. & Freedman, B. Diesel fuel production and qualities from vegetable oil. Fuel, 1987, 66, 1372-1378), more frequently also acids, salts or enzymes.

Reactions using the catalysts are usually pretentious regarding the quality and purity of the parent substances since the parent substances may contain additives which may affect the catalyst. The side reactions like the hydrolysis of the esters, saponification or other may occur to some extent. In addition, those reactions might not reach completely to the end and therefore part of the initial esters remain unchangeable. Yields of the side reactions or initial esters remaining unreacted may essentially encumber the separation and purification of reaction yields.

There are also known the carboxylic acid transesterification methods in the supercritical environment. For example the USA patent document U.S. Pat. No. 6,187,939 describes the method of obtaining fatty acid esters and fuel from fatty acid esters.

Among the well-known solutions, there is no information on continuous process carboxylic acid ester transesterification method in supercritical monovalent alcohol which could be appropriate for industrial use. The continuous process transesterification reactions have been described e.g. in the article by Noureddini, H., Harkey, D. & Medikonduru, V. A.—Continuous process for vegetable oil conversions to fatty acid methyl esters. J. Am. Chem. Soc., 1998, 75, 1775-1783.

USA patent document U.S. Pat. No. 6,262,294 describes the continuous process method for obtaining the divalent aromatic carboxylic acid esters in reaction with aromatic polyesters and supercritical alcohol.

From the well-known solutions the closest to the present inventions might be regarded the European patent documents EP 0985654 and EP 1298192, which describe the fatty acid transesterification methods in supercritical alcohol with the aim to get the compounds suitable for use as fuels or fuel additives.

European patent document EP 0985654 describes the method for producing fatty acid esters and fuel containing fatty acid esters. According to that method at least one of fatty acids, oils and alcohol involved in the reaction is in supercritical state or the alcohol is in supercritical state. Pressure of the reaction environment does not exceed 25 MPa. Alcohol corresponds to the formula R—OH where R is hydrocarbyl group having 1 to 10 carbon atoms or hydrocarbyloxyl group substituted by the hydrocarbyl group having 2 to 10 carbon atoms altogether or methyl group or ethyl group. Different fats, oils and their residues involved in the reaction are used as parent substances. Fuel, diesel oil, lubricant or fuel additive is the yield containing the fatty acid ester produced by that method.

The European patent document EP 1298192 describes the method and device for producing fatty acid esters. According to that method related reaction the monohydrid alcohol involved with fats and oils is in supercritical state. Reaction mixture contains unreacted substances and/or intermediate products which are in circulation in the reactor. Fatty acid separation from the reaction mixture. Monohydrid alcohol corresponds to the general formula R—OH where R is hydrocarbyl group having 1 to 10 carbon atoms or hydrocarbyloxyl group substituted by hydrocarbyl group having 2 to 10 carbon atoms altogether or methyl group or ethyl group—the device has a mechanism for reusing the unreacted substances and/or intermediate compounds in the reactor—the device has the means for separating the fatty acid esters from reaction mixture.

But in that case the quality and yield of the products is not good enough, in addition they contain unreacted parent substances and intermediate products. Electricity is used for heating which is not very efficient.

### DISCLOSURE OF INVENTION

The aim of the present invention is to offer a solution different from the well-known ones for continuous process carboxyl acid esters transesterification device for industrial use and relevant method for easy use in case of big volumes and free from the above mentioned shortcomings.

The nature of the method corresponding to the present invention is obtaining new carboxylic acids (incl. fatty acids) esters as a continuous process in the supercritical monovalent alcohol proceeding from the natural or synthetic carboxylic acid esters, e.g. fats and fat oils which are the esters of different fatty acids and glycerol.

For implementing the aim corresponding to the invention, the device containing alcohol and carboxylic acid ester inlets, reactant dosing unit, heat exchanger, transesterification device, hot reaction yield tube to expansion tank, expansion tank, tube of hot reaction yield to heat-exchanger, reverse current tube of excess alcohol, tube of cooled reaction yield to separator, separator, outlets of new ester and new alcohol resulting from the process is used.

The transesterification device comprises the pump, heat source, reactor with the shape of a tube for example and an outlet valve. The transesterification reaction is performed in the tube-shaped reactor where the conditions necessary for the performance of reaction are guaranteed. It is important from the viewpoint of the reaction that alcohol, received as one of the parent substance components from the reactor inlet, turns into supercritical state when reaching the reactor.

The carboxylic acid esters, being the raw material, change in the transesterification process in the reactor into new esters and alcohol in the ester molecule releases.

Transesterification reactions are the common name for the organic reaction group in the course of which the alcoxy groups in the ester molecule are replaced by the groups of other alcohol alcoxy groups. In case of the reactions corresponding to the present invention they are the balance reactions proceeding according to the transesterification general reaction equation

R²—COOR³+R¹—OH→R²—COOR¹+R³—OH

as follows:

M[-O(O)C—R^{x}]ₙ+nR¹—OH→nR^{x}—COOR¹+M[OH]ₙ,
where R¹ marks the aliphatic hydrocarbon radical having 1 to 10 carbon atoms; R², R³ and R^{x} mark aliphatic hydrocarbon radicals having 2 to 30 carbon atoms; n is a number in the range of 1-6; M marks aliphatic or alicyclic multivalent radical containing 0 to 6−n (6 minus n) OH groups.

Carboxyl acid esters used as parent substances may be natural as well as synthetic or their mixtures. Vegetable oils or animal fats or other compounds with natural origin may be the natural esters of carboxyl acids. Different chemical industry products containing at least one aliphatic carboxyl acid residue connected with ester link may be the synthetic ester. For example rapeseed oil, sunflower oil, soya oil, palm oil, lard, animal fat, fish fat are such kind of natural carboxyl acid esters. Free carboxyl acids, alcohols, vitamins, steroids, colorants, light and heavy naphtha products may occur as additives in the carboxyl acid esters used as parent substances.

Alcohol used in transesterification process corresponds to general formula R—OH where R marks any aliphatic hydrocarbon radical. For example methanol, ethanol, propanol, butanol and their any kind of isomers (structural and optical) satisfy those conditions. The substance is in the supercritical state when the critical temperature and critical pressure of that substance have been exceeded.

Table 1 below gives the critical temperatures (T_{critical}) and pressures (P_{critical}) Of alcohols used in the reaction and corresponding to the method.

TABLE 1

| | T_{critical} ° [C.] | P_{critical} ° [MPa] |
|---|---|---|
| Methanol | 239.45 | 8.1 |
| Ethanol | 240.75 | 6.15 |
| n-Propanol | 263.65 | 5.18 |
| i-Propanol | 235.15 | 4.76 |
| n-Butanol | 289.95 | 4.42 |
| 2-Butanol | 290 | 4.18 |
| i-Butanol | 275 | 4.3 |
| Tert-Butanol | 233 | 3.97 |
| n-Penthanol | 315 | 3.91 |
| n-Hexanol | 338.25 | 3.51 |
| Glycerol | 452.85 | 6.68 |

The properties of the substance in supercritical state differ essentially from the properties of the substance under normal conditions by several features. The viscosity of the substance in supercritical state is close to the viscosity of gas and density close to the density of liquid. Heat conductivity and diffusion ability features remain between the relevant features of the liquid and gaseous state. Reaction ability of a substance in the supercritical state or a substance diluted in it is higher than in gaseous or liquid state.

Carboxylic acids ester transesterification reaction according to this invention is performed as a continuous process in a tube-shaped reactor. During the whole operation of the reactor there should be necessary conditions for the successful realization of the reaction—environmental pressure, temperature, molar ratio of the reactants an time during which the reactants should stay in the given environment.

In the terms of this invention the most important part of the transesterification device is a tube-shaped reactor where it is possible to create and keep necessary conditions for continuous process transesterification of carboxylic acids in the supercritical monovalent alcohol. Besides the reactor the other important parts of the transesterification device is a pump, heat source and outlet valve.

The pump ensures the working pressure of the reactor and continuous flow of the reactants. The output pressure of the pump should be 1-5% higher than the minimum needed for opening the outlet valve. The output power of the pump in a minute depends on the capacity of the reactor tube, time determined for performing the reaction and the speed of heating the reactants. For calculating the output power of the pump the following formula 1 may be used:

P=V/(Vr+Vk),

where P is the output power of the pump,

V is the capacity of the reactor's tube,

Vr is the speed of reaction,

Vk is the speed of heating.

At the temperature of 350° C. and more and in case of the molar ratio 1/15 the output power of the pump in a minute is no more than ½ of the capacity of the reactor. For example in case of the reactor with the capacity of 40 l the power of the pump should not exceed 20 l/min.

Tube made from the hot and pressure-proof material form the main part of the reactor resisting the temperature determined for performing the reaction and pressure needed for it at least by 1.7 times. The structure of the reactor should ensure the sound stay and forward movement of the reactants for performing the reaction during the minimum time. One possibility to ensure the stay in the reactor during a certain time and the movement of the reactants is to use a tube with an appropriate length and diameter ratio. In that case the length of the tube is preferably at least 5 times bigger than the diameter. Depending on the need the tube may also be rolled or square or other. Second possibility is to fill the suitable cylinder with honeycomb wise flow directional partitions. In that case the ratio of the length of the tube and diameter is not definitely determined.

The mixture of reactants given to the reactor inlet is heated in the first third of the tube until the intended temperature. The outlet of the reactor is closed with a valve ensuring that the pressure needed for performing the reaction stays on the required level. The opening pressure of the valve equals to the one determined for performing the reaction and is accordingly 1-5% lower from the output pressure of the pump.

Reactor tube can be, depending on the need, heated from inside or outside. According to this invention, microwaves, electricity or gas or other is used as the heat source. Preferably the microwaves are used as this is the most efficient possibility of heating.

### MODE OF INVENTION

The method and device with references to the attached flow chart of the device corresponding to the invention is described below.

The device depicted on the drawing comprises of alcohol inlet 1, carboxylic acid ester inlet 2, reactant dosing unit 3, heat exchanger 4, transesterification device 5, hot reaction yield tube to expansion tank 6, expansion tank 7, hot reaction yield tube to heat expansion 8, reverse current tube of excess 9, tube of cooled reaction yield tube to separator 10, separator 11, new ester and new alcohol outlets resulting from the process 12 and 13.

Transesterification device 5 comprises of pump 14, heat source 15, tube-shaped reactor for performing the transesterification reaction 16, outlet valve 17.

Alcohol received from inlet 1 and carboxylic acid ester received from inlet 2 are mixed in a proper ratio in the dosing unit 3. The minimum ester-alcohol ratio is 2/1 with two mass parts of ester and 1 mass part of alcohol, preferred ratio is 1/3.

When the molecule weight of the initial ester is higher than 400, calculations of ester/alcohol proportion may be done according to the following equation (molecule mass of initial ester)/(number of ester links in the ester molecule*5*molecule mass of alcohol)=mass of initial ester/mass of alcohol.

The molar ratio of the parent substances—alcohol and carboxyl acids esters means that for the ester link in 1 ester molecule there are at least 2 alcohol molecules. Preferred molar ratio is 1/5 even more preferred is molar ratio 1/15.

The alcohol and carboxyl acid ester mixture moves from the dosing unit 3 to the heat exchanger 4 where the preheating takes place. The pre-heated mixture moves from heat exchanger 4 to the pump 14 of the transesterification device 5 where their pressure is risen to the determined level and directed to heat source 15.

The pressure of the reaction environment should be 0.5 MPa higher than the critical pressure of the used alcohol and it should not remain below 25 MPa. In the interest of the reaction speed and yield the pressure is kept preferably over 35 MPa. After reactor 16 there is no upper limit of the endurance to the pressure.

The temperature of the parent substances will be risen to the necessary level in the heat source 15, thereafter the mixture moves to reactor 16 where it stays for a determined time. Minimum time between 1.5-60 min is necessary for performing the reaction. Preferably it remains between 2-4 minutes. According to the molar ratio, in case of 1/5 molar ratio the time for the reaction mixture to stay in the reactor is up to 8 minutes, in case of the 1/15 molar ratio the time of the reaction mixture to stay in the reactor is up to 2 minutes.

Reaction speed depends on the composition of carboxylic acid esters and alcohol used as the parent substances, ambient temperature and pressure. Usually the increase of ambient temperature or pressure increase the reaction speed.

As the heat source 15 and reactor 16 form part of the transesterification device 5, the increase of temperature occurs at the same time with the forward movement of the reaction mixture. The temperature exceeds the critical temperature of the alcohol used in the process and is at least 300° C. There is no direct upper limit set for the reaction environment temperature but preferably it should not exceed 450° C., since at higher temperatures the pyrolysis reactions start occurring to some extent which decrease the quality and yield of the reaction. The preferred temperature of the reaction environment remains in the range of 350° C.-450° C.

After a determined time the reaction yields leave from the outlet valve 17 and move through tube 6 to the expansion tank 7 where the pressure drops to 0,1-1 MPa. The hot reaction yields move from expansion tank 7 along the tube 8 to the heat exchanger 4. Along the tube 9 the alcohol, having separated in a gaseous state in expansion tank 7 as the result of pressure drop, is directed back to the beginning of the process. The reaction yields move from heat exchanger 4 through the tube 10 to the separator 11 where new esters and new alcohol is separated which leave accordingly from outlets 12 and 13.

### EXAMPLE 1

When methanol and rapeseed oil are used as parent substances then according to the method corresponding to the invention the alcohol is conveyed to supercritical state where the temperature of methanol is at least 239.45° C. and pressure is 8.084 MPa. For that at least 15 molecules of alcohol are taken per 1 molecule of rapeseed oil. In preferred realization at least 45 molecules of alcohol are taken per 1 molecule of rapeseed. The reaction time at the temperature of 350° C. and pressure of 45 MPa is below 2 minutes.

Methanol (98%) and rapeseed oil are mixed in the ratio of 3 mass parts of methanol per 1 mass part of oil and after receiving the mixture the temperature is increased to 350° C. and pressure to 45 MPa and the mixture flows on those conditions through the reactor with the capacity of 3 l with the speed of 1.5 l/min. The mixture leaving the reactor contains rapeseed methylester, glycerole and unreacted methanol.

### EXAMPLE 2

Ethanol (95%) and rapeseed oil are mixed in the ration of 4 mass parts of ethanol per 1 mass part of oil and after receiving the mixture the temperature is increased to 350° C. and pressure to 45 MPa and the mixture flows on those conditions through the reactor with the capacity of 3 l with the speed of 1.5 l/min. The mixture leaving the reactor contains rapeseed ethylester, glycerole and unreacted ethanol.

### EXAMPLE 3

Ethandioldibuthyrate and methanol are mixed with methanol in the ratio of 1/3. The received mixture is heated with 350° C. and pressure increased to 45 MPa and directed through the reactor with the capacity of 3 litres with the speed of 1.5 l/min. The mixture leaving the reactor contains methylbuthyrate, ethandiol and unreacted methanol.

For the specialists of the relevant field it is obvious that the possibilities of the given device and method are not limited only with the examples given in the performance examples.

1. Method for continuous process transesterification of carboxylic acid esters in supercritical monovalent alcohol corresponding to the general formula R—OH where R indicates any aliphatic hydrocarbon radical having 1 to 10 carbon atoms and which is industrially usable and the parent substances of which are natural, synthetic carboxylic acid esters or their mixtures being for example fats and fat oils which are the esters of different fatty acids and glycerols, characterized in that
the alcohol received from inlet (1) and carboxyl acid ester received from inlet (2) are mixed together in the dosing unit (3), since in molar ratio there are at least two alcohol molecules per ester link in one ester molecule and since the mass part ratio of ester and alcohol is at least 2/1,
the alcohol and carboxyl acid ester mixture is taken from the dosing unit (3) for pre-heating to the heat exchanger (4),
the pre-heated mixture is directed from the heat exchanger (4) to transesterification device (5) pump (14) where the pressure of the reaction environment of the mixture is increased to the pressure of at least 25 MPa, whereas the pressure of the reaction environment is 0.5 MPa higher than the critical pressure of the used alcohol,
thereafter the mixture is directed to the heat source (15) where the temperature of the reaction environment is increased to at least 300° C., whereas the temperature of the reaction environment exceeds the critical temperature of alcohol used in the process,
the mixture is directed further to the tube-shaped reactor (16),
after the reaction time passes, the reaction yields are directed through the outlet valve (17) and tube (6) to the expansion tank (7) where the pressure is dropped to 0.1-1 MPa,
the hot reaction yields are taken from the expansion tank (7) along the tube (8) to the heat exchanger (4),
then along the tube (9) the alcohol having separated in a gaseous state in the expansion tank (7) is directed back to the beginning of the process,
the reaction yields are taken from the heat exchanger (4) through the tube (10) to the separator (11) where the new esters and new alcohol are separated leaving from relevant outlets (12) and (13).

2. Method as claimed in claim 1, characterized in that the transesterification reaction is performed according to the equation:
M[-O(O)C—R^{x}]ₙ+nR¹—OH→nR^{x}—COOR¹+M[OH]n,
where R¹ indicates the aliphatic hydrocarbon radical having 1 to 10 carbon atoms, R^{x} indicates aliphatic hydrocarbon radicals having 2 to 30 carbon atoms, n is a number in the range of 1-6; M marks aliphatic or alicyclic multivalent radical containing 0 to 6−n (6 minus n) OH groups.

3. Method as claimed in claims 1 and 2, characterized in that there are preferably five alcohol molecules per ester link involved in one parent substance molecule.

4. Method as claimed in claims 1 and 2, characterized in that the ratio of ester and alcohol mass unit is preferably 1/3.

5. Method as claimed in claim 1, characterized in that the pressure of the reaction environment is preferably kept over 35 MPa.

6. Method as claimed in claim 1, characterized in that the temperature of the reaction environment is preferably within the range of 350° C.-450° C.

7. Method as claimed in claim 3, characterized in that there are even more preferably fifteen alcohol molecules per ester link involved in the parent substance molecule.

8. Method as claimed in claims 2 and 6, characterized in that the microwaves are used for heating.

9. Device for the continuous process transesterification of carboxylic ester in supercritical monovalent alcohol which would be used industrially and comprises the alcohol inlet (1), carboxylic acid ester inlet (2), reactant dosing unit (3), heat exchanger (4), transesterification device (5), hot reaction yield tube to expansion tank (6), expansion tank (7), tube of hot reaction yield to heat-exchanger (8), reverse current tube of excess of alcohol (9), tube of cooled reaction yield to separator (10), separator (11), outlets of new ester and new alcohol resulting from the process (12 and 13), characterized in that the transesterification device (5) comprises the pump (14), the heat exchanger (15), tube-shaped reactor (16), outlet valve (17) and that the tube-shaped reactor (16) is closed with outlet valve (17), the output power of the pump (14) is 1-5% higher than the minimum necessary for opening the outlet valve (17), tube-shaped reactor (16) bears the temperature determined for the performance of the reaction and pressure needed for that at least for 1.7 times; outlet valve (17) opening pressure equals to the determined one and is 1-5% lower from the output power of the pump (14).

10. Device as claimed in claim 8, characterized in that the length of the tube-shaped reactor (16) is preferably at least 5 times bigger than the diameter.
